(19) United States
(12) Patent Application Publication
(10) Pub. No.: US 2003/0105378 A1
(43) Pub. Date: Jun. 5, 2003

(54) Process for recovery of diene-free feedstocks from olefinic process streams

(76) Inventors: Larry R. Satek, St. Charles, IL (US); Michael J. Foral, Aurora, IL (US); Diadema N. Ventura, Lisle, IL (US)

Correspondence Address: CAROL WILSON, BP AMERICA INC., MAIL CODE 5 EAST, 4101 WINFIELD ROAD, WARRENVILLE, IL, 60555, US

(21) Appl. No.: 09/997,863

(22) Filed: Nov. 30, 2001

(51) Int. Cl. C07C 7/12

(52) U.S. Cl. 585/820; 585/810; 585/829; 585/826

(57) ABSTRACT

Processes using heterogeneous adsorbents are disclosed for purification of olefins such as are typically produced by thermal cracking of suitable hydrocarbon feedstocks. The processes for recovery of diene-free feedstocks includes passing an olefinic process stream containing undesirable levels of propadiene, and optionally hydrocarbon compounds having more than one double bond, small amounts of acetylenic impurities, and/or other organic components, through a particulate bed of heterogeneous adsorbent comprising a metal supported on a high surface area carrier, under conditions suitable for adsorption of dienes. Beneficially, the resulting gaseous mixtures also have reduced levels of other hydrocarbons having more than one double bond, and have reduced levels of acetylenic impurities, such as acetylene and methylacetylene. Processes according to this invention are particularly useful where the olefin being purified is ethylene and/or propylene formed by thermal cracking of hydrocarbon feedstocks from the adsorbent.

### FIELD OF THE INVENTION

The field of this invention relates to use of heterogeneous adsorbents in purification of relatively impure olefins such as are typically produced by thermal cracking of suitable hydrocarbon feedstocks. More particularly, this invention concerns recovery of diene-free feedstocks by passing an olefinic process stream containing undesirable levels of propadiene, and optionally hydrocarbon compounds of from 4 to about 6 carbon atoms having more than one double bond, small amounts of acetylenic impurities, and/or other organic components, through a particulate bed of heterogeneous adsorbent comprising a metal supported on a high surface area carrier, under conditions suitable for adsorption of dienes. Beneficially, the resulting gaseous mixtures also have reduced levels of other hydrocarbons having more than one double bond, and have reduced levels of acetylenic impurities, such as acetylene and methylacetylene. Processes according to this invention are particularly useful where the olefin being purified is ethylene and/or propylene formed by thermal cracking of hydrocarbon feedstocks.

### BACKGROUND OF THE INVENTION

As is well known, olefins, or alkenes, are a homologous series of hydrocarbon compounds characterized by having a double bond of four shared electrons between two carbon atoms. The simplest member of the series, ethylene, is the largest volume organic chemical produced today. Olefins including, importantly, ethylene, propylene and smaller amounts of butadiene, are converted to a multitude of intermediate and end products on a large scale, mainly polymeric materials. Furthermore, sources of relatively impure olefins may also contain undesirable levels of hydrocarbons having more than one double bond, for example dienes such as propadiene, 1,2-butadiene, 1,3-butadiene, 1,2-pentadiene, 1,3-pentadiene, 2,3-pentadiene, 2-methyl-1,3-butadiene (isoprene), and cyclopentadiene and/or trienes such as 1,3,5-hexatriene.

Commercial production of olefins is, almost exclusively, accomplished by pyrolysis of hydrocarbons in tubular reactor coils installed in externally fired heaters. Thermal cracking feed stocks include streams of ethane, propane or a hydrocarbon liquid ranging in boiling point from light straight-run gasoline through gas oil. Because of the very high temperatures employed, commercial olefin processes invariably coproduce significant amounts of acetylene, methyl acetylene, and propadiene. Required separation of the more highly unsaturated contaminating compounds from the primary olefin can, considerably, increase the plant cost.

In a typical ethylene plant the cracking section represents about 25 percent of the cost of the unit while the compression, heating, dehydration, recovery and refrigeration sections represent the remaining about 75 percent of the total. This endothermic process is carried out in large pyrolysis furnaces with the expenditure of large quantities of heat which is provided in part by burning the methane produced in the cracking process. After cracking, the reactor effluent is put through a series of separation steps involving cryogenic separation of products such as ethylene and propylene. The total energy requirements for the process are thus very large and ways to reduce it are of substantial commercial interest. In addition, it is of interest to reduce the amount of methane and heavy fuel oils produced in the cracking processor to utilize it other than for its fuel value.

Hydrocarbon cracking is carried out using a feed which is ethane, propane or a hydrocarbon liquid ranging in boiling point from light straight-run gasoline through gas oil. Ethane, propane, liquid naphthas, or mixtures thereof are preferred feed to a hydrocarbon cracking unit. Hydrocarbon cracking is, generally, carried out thermally in the presence of dilution steam in large cracking furnaces which are heated by burning, at least in part, methane and other waste gases from the olefins process resulting in large amounts of NOx pollutants. The hydrocarbon cracking process is very endothermic and requires large quantities of heat per pound of product. However, newer methods of processing hydrocarbons utilizes at least to some extent catalytic processes which are better able to be tuned to produce a particular product slate. The amount of steam used per pound of feed in the thermal process depends to some extent on the feed used and the product slate desired. Typically, steam pressures are in the range of about 30 lbs per sq in to about 80 lbs per sq in, and amounts of steam used are in the range of about 0.2 pounds of steam per pound of feed to 0.7 pounds of per pound of feed. The temperature, pressure and space velocity ranges used in thermal hydrocarbon cracking processes to some extent depend upon the feed used and the product slate desired which are well known as may be appreciated by one skilled in the art.

The type of furnace used in the thermal cracking process is also well known. However the ceramic honeycomb furnace which is described in U.S. Pat. No. 4,926,001, the contents of which patent are specifically incorporated herein by reference, is an example of a new type of cracking which could have a special utility for this process.

Several methods are known for separation of an organic gas containing unsaturated linkages from gaseous mixtures. These include, for instance, cryogenic distillation, liquid adsorption, membrane separation and the so called “pressure swing adsorption” in which adsorption occurs at a higher pressure than the pressure at which the adsorbent is regenerated. Cryogenic distillation and liquid adsorption are common techniques for separation of carbon monoxide and alkenes from gaseous mixtures containing molecules of similar size, e.g., nitrogen or methane. However, both techniques have disadvantages such as high capital cost and high operating expenses. For example, liquid adsorption techniques suffer from solvent loss and need a complex solvent make-up and recovery system.

Molecular sieves which selectively adsorb carbon monoxide from gaseous mixtures by chemisorption are also known. U.S. Pat. No. 4,019,879 and U.S. Pat. No. 4,034,065 refer to use of high silica zeolites, which have relatively high selectivities for carbon monoxide, in the pressure swing adsorption method. However, these zeolites only have moderate capacity for carbon monoxide and more particularly require very low vacuum pressures to recover the adsorbed gases and/or to regenerate the zeolite.

U.S. Pat. No. 4,717,398 describes a pressure swing adsorption process for selective adsorption and subsequent recovery of an organic gas containing unsaturated linkages from gaseous mixtures by passing the mixture over a zeolite ion-exchanged with cuprous ions (Cu I) characterized in that the zeolite has a faujasite type crystalline structure (Y).

Kokai JP Number 50929-1968 describes a method of purifying vinyl compounds containing up to about 10 percent by weight of acetylene compounds including ethyl acetylene, vinyl acetylene and phenyl acetylene whereby the acetylene compounds are adsorbed in an adsorption agent of 1-valent and/or 0-valent copper and/or silver supported on inert carrier such as δ-alumina, silica or active carbon. However, it is well known that acetylene and these acetylene compounds react with copper and/or silver to from copper acetylide or silver acetylide. Both the acetylide of copper and silver are unstable compounds. Because they are explosive under some conditions their possible formation presents safety problems in operation and in handling adsorbent containing such precipitates.

German Disclosure Document 2059794 describes a liquid adsorption process for purification of paraffinic, olefinic and/or aromatic hydrocarbons with an adsorption agent consisting in essence of a complex of a copper (Cu I)-salt with an alkanolamine such as mono-ethanolamine, mono-isopropanolamine, di-ethanolamine, tri-ethanolamine and arylalkanolmines, and optionally in the presence of a glycol or polyglycol. However, the product stream is contaminated with unacceptable levels of components of the such agents absorbed in the hydrocarbon flow. While such contamination might be removable using an additional bed of silica gel, aluminum oxide or a wide-pored molecular sieve, this would involve additional capital costs, operation expenses and perhaps safety problems.

Processes using heterogeneous adsorbents are known for purification of olefins, such as are typically produced by thermal cracking of suitable hydrocarbon feedstocks, by passing a stream of olefin through a particulate bed of support material on which is dispersed a metallic element. U.S. Pat. No. 6,080,905 and U.S. Pat. No. 6,124,517 in the name of Mark P. Kaminsky, Shiyou Pei, Richard A Wilsak, and Robert E. Whittaker describe adsorption which is carried out in an essentially dihydrogen-free atmosphere within the bed. Adsorption of the contained acetylenic impurities is continued until levels of acetylenic impurities in the effluent stream increase to a predetermined level. Thereafter the resulting bed of adsorbent is regenerated using hydrogen to effect release of the contained acetylenic impurities from the adsorbent. However, there remains a need to increase the capacity of adsorbents for acetylenics whereby the useful life of the adsorbent bed between regenerations is increased.

More recently U.S. Pat. No. 6,215,037 in the name of Joel Padin, Curtis L. Munson and Ralph T. Yang provides a selection of specific zeolites said to be useful for selective adsorption of dienes from mono-olefins. In particular, the adsorbents are ion-exchanged zeolites of the group consisting of zeolite X, Zeolite Y and zeolite LSX in a form having exchangeable cationic sites. According to the patent, essentially all cationic sites of the ion-exchanged zeolite must contain silver cation or copper cation for the selective separation of diene from mono-olefin which the same number of carbon atoms.

Olefin-paraffin separations represent a class of most important and also most costly separations in the chemical and petrochemical industry. Cryogenic distillation has been used for over 60 years for these separations. They remain to be the most energy-intensive distillations because of the close relative volatilities. For example, ethane-ethylene separation is carried out at about −25° C. and 320 pounds per square inch gauge pressure (psig) in a column containing over 100 trays, and propane-propylene separation is performed by an equally energy-intensive distillation at about −30° C. and 30 psig.

Impurity refers to compounds that are present in the olefin plant feedstocks and products. Well-defined target levels exist for impurities. Common impurities in ethylene and propylene include: acetylene, methyl acetylene, methane, ethane, propane, propadiene, and carbon dioxide. Listed below are the mole weight and atmospheric boiling points for the light products from thermal cracking and some common compounds potentially found in an olefins unit.

| | Mole | Normal Boiling |
|---|---|---|
| Compound | Weight | Point, ° C. |
| Hydrogen | 2.016 | −252.8 |
| Nitrogen | 28.013 | −195.8 |
| Carbon monoxide | 28.010 | −191.5 |
| Oxygen | 31.999 | −183.0 |
| Methane | 16.043 | −161.5 |
| Ethylene | 28.054 | −103.8 |
| Ethane | 30.070 | −88.7 |
| Phosphine | 33.970 | −87.4 |
| Acetylene * | 26.038 | −84.0 |
| Carbon dioxide * | 44.010 | −78.5 |
| Radon | 222.00 | −61.8 |
| Hydrogen sulfide | 34.080 | −60.4 |
| Arsine | 77.910 | −55.0 |
| Carbonyl sulfide | 60.070 | −50.3 |
| Propylene | 42.081 | −47.8 |
| Propane | 44.097 | −42.1 |
| Propadiene (PD) | 40.065 | −34.5 |
| Cyclo-propane | 42.081 | −32.8 |
| Methyl acetylene | 40.065 | −23.2 |
| Water | 18.015 | 100. |

* Sublimation temperature

Included are some compounds which have similar boiling temperatures to cracked products and may be present in feedstocks or produced in trace amounts during thermal cracking.

Polymer grades of ethylene and propylene must contain very low amounts of acetylene, methylacetylene and propadiene. These compounds are known to have a negative impact on polymers produced with Ziegler-Natta catalysts and chromium based catalysts, and they are particularly bad for the new metallocene catalysts where they are believed to destroy catalyst molecules stoichiometrically. Thus technologies that can produce olefins free of these materials are potentially very valuable.

Recently the trend in the hydrocarbon processing industry is to reduce commercially acceptable levels of impurities in major olefin product streams, i.e., ethylene, propylene, and hydrogen. Need for purity improvements is directly related to increasing use of higher activity catalysts for production of polyethylene and polypropylene, and to a limited extent other olefin derivatives.

Typically, acetylene is the predominant impurity and is hydrogenated to either ethylene or ethane during the course of the reaction to low levels with the aid of palladium catalysts and large amounts of hydrogen. However, methylacetylene and propadiene are not completely hydrogenated to propane or propylene in ethylene streams unless very severe conditions are imposed, resulting in significant ethylene losses. Since they are formed in lower levels, they are removed by other means. Other technologies may produce larger amounts of methylacetylene or propadiene. Thus simple hydrogenation may not be acceptable, and a technology which can selectively remove these impurities with no product loss may have a role in the commercialization of the new olefin technology.

It is known that acetylenic impurities can be selectively hydrogenated and thereby removed from such product streams by passing the product stream over an acetylene hydrogenation catalyst in the presence of dihydrogen (molecular hydrogen, H₂). However, these hydrogenation processes typically result in the deposition of carbonaceous residues or “green oil” on the catalyst which deactivates the catalyst. Therefore, acetylene hydrogenation processes for treating liquid or liquefiable olefins and diolefins typically include an oxygenation step or a “burn” step to remove the deactivating carbonaceous residues from the catalyst followed by a hydrogen reduction step to reactivate the hydrogenation catalyst. For example, see U.S. Pat. No. 3,755,488 to Johnson et al., U.S. Pat. No. 3,792,981 to Hettick et al., U.S. Pat. No. 3,812,057 to Morgan and U.S. Pat. No. 4,425,255 to Toyoda. However, U.S. Pat. No. 3,912,789 and U.S. Pat. No. 5,332,705 state that by using selected hydrogenation catalysts containing palladium, at least partial regeneration can be accomplished using a hydrogenation step alone at high temperatures (600° F.-700° F.) and in the absence of an oxygenation step.

Selective hydrogenation of the about 2000 to 4000 parts per million of acetylenic impurities to ethylene is, generally, a crucial operation for purification of olefins produced by thermal steam cracking. Typical of a small class of commercially useful catalysts are materials containing very low levels of an active metal supported on an inert carrier, for example a particulate bed having less than about 0.03 percent (300 ppm) palladium supported on the surface skin of carrier pellets having surface area of less than about 10 m²/gm.

Many commercial olefin plants using steam crackers use, generally, front-end acetylene converters, i.e., the hydrogenation unit is fed C₃ and lighter cracked gas which feed has a high enough concentration of hydrogen to easily hydrogenate the acetylenic impurities, however, when run improperly, will also hydrogenate a large fraction of the ethylene and propylene product. Both hydrogenation of acetylene and ethylene are highly exothermic.

Accelerated catalyst deactivation and thermal runaways caused by loss in catalyst selectivity are common problems which plague acetylene converters. Such problems result in unscheduled shutdowns and increased costs to replace deactivated catalyst.

The problem of over-hydrogenation is aggravated because the rate constant for ethylene hydrogenation to ethane is 100 times faster than for the hydrogenation of acetylene to ethylene. As a means to avoid a C₂H₄ hydrogenation thermal runaway, acetylene, carbon monoxide and diolefins concentrations must, therefore, be high enough to cover most of the active sites so that none are left to adsorb ethylene. For example, acetylene, carbon monoxide, methyl acetylene, and propadiene have bond strengths to palladium which are stronger than the ethylene to palladium bonds. Selection of active metal, size of the metal particles and other physical and chemical factors ultimately affect the “operating temperature window” which is the delta of temperature between acetylene conversion to ethylene (typically in a range from about 100° F. to about 150° F.) and thermal runaway where all molecular hydrogen is converted and a large amount of the ethylene is converted to ethane (about 170° F. to about 225° F.). The wider the window, the safer is operation of the unit.

It is therefore a general object of the present invention to provide an improved process which overcomes the aforesaid problem of prior art methods, for production of olefins from thermal cracking of hydrocarbon feed stocks which olefin can be used for manufacture of polymeric materials using higher activity catalysts.

More particularly, it is an object of the present invention to provide an improved method for purification of ethylene and/or propylene containing undesirable levels of propadiene, and optionally hydrocarbon compounds of from 4 to about 6 carbon atoms having more than one double bond, and/or other organic components that are impurities in olefinic process streams, by passing the impure olefin stream through a particulate bed of heterogeneous adsorbent comprising a metal supported on a high surface area carrier, under conditions suitable for adsorption of the impurities having more than one double bond.

It is another object of the present invention to provide an improved aforesaid purification method that employs an adsorbent that, even after a substantial period of aging, exhibits ability to withstand repeated regenerations and yet retain useful adsorption capacity.

Other objects and advantages of the invention will become apparent upon reading the following detailed description and appended claims.

### SUMMARY OF THE INVENTION

Economical processes are disclosed for purification of a relatively impure olefins produced by thermal cracking of hydrocarbons. Processes of this invention comprise: providing a fluid mixture predominantly comprising at least one olefin of from 2 to about 8 carbon atoms, impurities comprising propadiene and optionally hydrocarbon compounds of from 4 to about 6 carbon atoms having more than one double bond and/or acetylenic impurities having the same or similar carbon content in an amount of up to about 1 percent by volume base upon the total amount of olefin present and optionally saturated hydrocarbon gases; passing the fluid mixture through a particulate bed of adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditions suitable for adsorption within the bed, to effect, in the presence of an essentially dihydrogen-free atmosphere within the bed, selective adsorption and/or complexing of the contained impurities with the adsorbent, and thereby obtain purified effluent which contains less than about 1 part per million by volume of the propadiene impurity; and thereafter regenerating the resulting bed of adsorbent in the presence of a reducing gas comprising dihydrogen to effect release of the contained impurities from the adsorbent.

In another aspect the invention is a process for purification of olefins produced by thermal cracking of hydrocarbons which comprises: passing a fluid mixture comprising at least about 50 percent by volume of an olefin having from 2 to about 4 carbon atoms, and impurities comprising propadiene and optionally hydrocarbon compounds of from 3 to about 6 carbon atoms having more than one double bond and/or acetylenic impurities having the same or similar carbon content in an amount in a range upward from about 1 to about 1000 parts per million by volume, through a particulate bed of adsorbent comprising predominantly a support material selected from the group alumina, silica, active carbon, clay and zeolites having surface area in a range of from about 10 to about 2,000 square meters per gram as measured by the BET gas adsorption method, on which is dispersed at least one metallic element selected from the group consisting of chromium, iron, cobalt, nickel, copper, ruthenium, palladium, silver and platinum, to provide an effluent stream from the bed; effecting, in the presence of an essentially dihydrogen-free atmosphere within the bed, selective and reversible adsorption and/or complexing of the contained diene and acetylenic impurities with the adsorbent, until levels of the diene and.or acetylenic impurities in the effluent stream increase to a predetermined level in a range downward from about 1 parts per million by volume; and thereafter regenerating the resulting bed of adsorbent in the presence of a reducing gas comprising dihydrogen to effect release of the contained impurities from the adsorbent.

Another aspect of special significance is the separation of diene and acetylenic impurities from ethylene or propylene containing small amounts of dienes and acetylene, i.e., less than about 5000 parts per million by weight of one or more acetylenic impurities, and provide, advantageously, purified product containing less than about 1 parts per million by weight, and frequently even less than about 0.5 parts per million by weight of the impurities.

In yet another aspect the invention is a process for purification of an olefinic stream to obtain a diene-free feedstock suitable for formation of polymeric resins, which purification process comprises: providing an impure gaseous stream comprising at least about 99 percent by volume of an olefin selected from the group consisting of ethylene and propylene, impurities comprising propadiene and optionally hydrocarbon compounds of from 3 to about 5 carbon atoms having more than one double bond and/or acetylenic impurities having the same or similar carbon content in an amount in a range upward from about 1 to about 1000 parts per million by volume based upon the total amount of olefin present and optionally saturated hydrocarbon gases; passing the impure stream through a bed of adsorbent which is free of a substantial amount of carbon monoxide, the adsorbent comprising at least about 90 weight percent of gamma alumina having surface area in a range of from about 150 to about 350 square meters per gram as measured by the BET gas adsorption method, on which is dispersed is at least one element selected from the group consisting of iron, cobalt, nickel, copper, palladium, silver and platinum, in the zero valent state, to effect, under conditions suitable for adsorption within the bed, selective adsorption and/or complexing of the contained impurities with the adsorbent, thereby obtain an effluent steam of feedstock which contains less than about 0.5 parts per million by volume of carbon monoxide and less than about 1 parts per million by volume of the dienes and acetylenic impurities; effecting, in the presence of an essentially dihydrogen-free atmosphere within the bed, selective adsorption and/or complexing of the contained dienes and acetylenic impurities with the adsorbent, until levels of the and acetylenic impurities in the effluent stream increase to a limiting level in a range downward from about 1 parts per million by volume; and thereafter regenerating the resulting bed of adsorbent in the presence of a reducing gas comprising dihydrogen which reducing gas is free of a substantial amount of carbon monoxide, to effect release of the contained impurities from the adsorbent.

A preferred class of adsorbents useful in processes according the invention, comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur. More preferred are the adsorbent which comprises at least about 90 weight percent of a gamma alumina having surface area in a range of from about 150 to about 350 square meters per gram as measured by the BET gas adsorption method, and wherein the metal dispersed on the support material is palladium, and the absorbent has a palladium content in a range of from about 0.01 to about 10 percent based on the total weight of the adsorbent.

For a more complete understanding of the present invention, reference should now be made to the embodiments illustrated in greater detail and described below by way of examples of the invention.

### BRIEF DESCRIPTION OF THE INVENTION

Processes of this invention are particularly suitable for use in purification of aliphatically unsaturated organic compounds produced, generally, by thermal cracking of hydrocarbons.

Unsaturated compounds of most interest with regard to purification by the method of the present invention, have two to about eight carbon atoms, preferably two to about four carbon atoms, and more preferably ethylene or propylene. Sources of desirable olefinic compounds may contain undesirable levels of hydrocarbons having more than one double bond, for example dienes such as propadiene, 1,2-butadiene, 1,3-butadiene, 1,2-pentadiene, 1,3-pentadiene, 2,3-pentadiene, 2-methyl-1,3-butadiene (isoprene), and cyclopentadiene and/or trienes such as 1,3,5-hexatriene.

The separation of propadiene from ethylene or propylene which may be contained in admixtures with acetylenic impurities and/or other normally gaseous materials, such as one or more of ethane, methane, propane and oxides of carbon is of particular importance. For example mixtures serving as a source of ethylene containing feed for the process may contain about 1 to about 99 weight percent ethylene, about 0 to about 50 weight percent ethane and/or about 0 to about 50 weight percent methane.

Generally acetylenic impurities described in this invention are expressed by the formula

R—C≡C—R

where R is hydrogen or a hydrocarbon group of up to 10 carbon atoms.

Optionally, it may be desired to treat the impure olefinic process stream used in the process of the present invention to remove any carbon monoxide. The amount of carbon monoxide in the fluid mixture should suitably be reduced to below 10 parts per million by weight, preferably below 2 parts per million by weight and most preferably below 1 parts per million by weight, prior to contact with the adsorbent. Similarly, it may be desirable to have low levels of dihydrogen in the olefinic feedstream to the adsorber for removal of contained diene impurities.

Any mercury-containing, arsenic-containing, and sulfur-containing components, e.g., hydrogen sulfide, present in the fluid mixture fed to the particulate bed of adsorbent should suitably be removed therefrom in any known manner in order to avoid the risk of poisoning the dispersed metal. The hydrocarbon mixture used in the process of the present invention is suitably a cracked gas from which the majority of the C₅ and higher hydrocarbons have been removed. The fluid mixture may thus comprise ethylene, propylene, butenes, methane, ethane, propane and butane.

In preferred embodiments of processes according to the invention, the olefin in the fluid mixture being purified is predominantly ethylene or propylene, the fluid mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range upward from about −78° C. to about 100° C., preferably in a range of from about −35° C. to about 65° C., and more preferably in a range of from about −10° C. to about 55° C.

The fluid mixture used in the process of the present invention may also comprise water and may optionally be saturated with water.

For processes according to invention the metal dispersed on the support material is, advantageously, at least one element selected from the group consisting of chromium, iron, cobalt, nickel, copper, ruthenium, palladium, silver and platinum, and the absorbent has a dispersed metal content in a range of from about 0.05 to about 20 percent based on the total weight of the adsorbent.

More preferred for processes according to this invention are adsorbents having palladium metal dispersed on the support, and the absorbent has a palladium content in a range of from about 0.05 to about 10 percent, more preferred palladium content in a range of from about 0.1 to about 5.0 percent based on the total weight of the adsorbent.

The adsorbent can, optionally, further comprise one or more elements selected from the group consisting of lithium, sodium, potassium, zinc, molybdenum, tin, tungsten, and iridium, dispersed on the support material. Preferably the adsorbent further comprises a member selected from the group consisting of lithium, sodium, potassium, zinc, molybdenum, and tin dispersed on the support material.

High metal dispersion and loading resulted in higher metal surface area. Capacity of an adsorbent is, typically, related directly to metal surface area. Any method which increases and/or maintains high metal surface area is, therefore, beneficial to achieving high adsorption capacity for dienes and acetylenic impurities.

Preferred for processes according to this invention are adsorbents having a dispersion value of at least about 10 percent, preferably in a range upward from about 20 percent to about 100 percent. Dispersion is a measure of the accessibility of the active metals on the adsorbent. Such dispersion methods are discussed in H. C. Gruber's, Analytical Chemistry, Vol. 13, p. 1828, (1962). The adsorbents for use in this invention were analyzed for dispersion using a pulsed carbon monoxide technique as described in more detail in the Examples. Palladium containing adsorbents having large dispersion values are desired because more of the palladium metal is available for adsorption.

Support materials are, advantageously, selected from the group consisting of alumina, silica, carbon, clay and zeolites (molecular sieves). Surface areas of support materials are, preferably, in a range of from about 10 to about 2,000 square meters per gram as measured by the BET gas adsorption method.

Generally, the term “molecular sieve” includes a wide variety of positive-ion-containing crystalline materials of both natural and synthetic varieties. They are generally characterized as crystalline aluminosilicates, although other crystalline materials are included in the broad definition. The crystalline aluminosilicates are made up of networks of tetrahedra of SiO₄ and AlO₄ moieties in which the silicon and aluminum atoms are cross-linked by the sharing of oxygen atoms. The electrovalence of the aluminum atom is balanced by the use of positive ions, for example, alkali-metal or alkaline-earth-metal cations.

Zeolitic materials, both natural and synthetic, useful herein have been demonstrated in the past to have catalytic capabilities for many hydrocarbon processes. Zeolitic materials, often referred to as molecular sieves, are ordered porous crystalline aluminosilicates having a definite structure with large and small cavities interconnected by channels. The cavities and channels throughout the crystalline material are generally uniform in size allowing selective separation of hydrocarbons. Consequently, these materials in many instances have come to be classified in the art as molecular sieves and are utilized, in addition to the selective adsorptive processes, for certain catalytic properties. The catalytic properties of these materials are also affected, to some extent, by the size of the molecules which are allowed selectively to penetrate the crystal structure, presumably to be contacted with active catalytic sites within the ordered structure of these materials.

In the past various molecular sieve compositions natural and synthetic have been found to be useful for a number of hydrocarbon conversion reactions. Among these are alkylation, aromatization, dehydrogenation and isomerization. Among the sieves which have been used are Type A, X, Y and those of the MFI crystal structure, as shown in “Atlas of Zeolite Structure Types,” Second Revised Edition 1987, published on behalf of the Structure Commission of the International Zeolite Associates and incorporated by reference herein. Representative of the last group are ZSM-5 and AMS borosilicate molecular sieves.

Prior art developments have resulted in the formation of many synthetic crystalline materials. Crystalline aluminosilicates are the most prevalent and, as described in the patent literature and in the published journals, are designated by letters or other convenient symbols. Exemplary of these materials are Zeolite A (Milton, in U.S. Pat. No. 2,882,243), Zeolite X (Milton, in U.S. Pat. No. 2,882,244), Zeolite Y (Breck, in U.S. Pat. No. 3,130,007), Zeolite ZSM-5 (Argauer, et al., in U.S. Pat. No. 3,702,886), Zeolite ZSM-11 (Chu, in U.S. Pat. No. 3,709,979), Zeolite ZSM-12 (Rosinski, et al., in U.S. Pat. No. 3.832,449), and others.

Manufacture of the ZSM materials utilizes a mixed base system in which sodium aluminate and a silicon containing material are mixed together with sodium hydroxide and an organic base, such as tetrapropylammonium hydroxide and tetrapropylammonium bromide, under specified reaction conditions, to form the crystalline aluminosilicate, preferably a crystalline metallosilicate exhibiting the MFI crystal structure.

A preferred class of molecular sieves useful, according to the present invention, are crystalline borosilicate molecular sieves disclosed in commonly assigned U.S. Pat. No. 4,268,420, U.S. Pat. No. 4,269,813, U.S. Pat. No. 4,292,457, and U.S. Pat. No. 4,292,458 to Marvin R. Klotz, which are incorporated herein by reference.

### BRIEF DESCRIPTION OF PREFERRED EMBODIMENTS OF THE INVENTION

While this invention is susceptible of embodiment in many different forms, this specification discloses only some specific forms as examples of the use of the invention. In particular, preferred embodiments of the invention for purification of a fluid mixture comprising olefin preferably an olefin of from two to about six carbon atoms having a single double bond, containing impurities including propadiene and optionally hydrocarbon compounds of from 3 to about 6 carbon atoms having more than one double bond and/or acetylenic impurities having the same or similar carbon content in an amount of up to about 1 percent by volume base upon the total amount of olefin present and optionally saturated hydrocarbon gases are illustrated and described. The invention is not intended to be limited to the embodiments so described, and the scope of the invention will be pointed out in the appended claims.

The apparatus of this invention is used with certain conventional components the details of which, although not fully illustrated or described, will be apparent to those having skill in the art and an understanding of the necessary function of such components.

More specifically an integrated olefin purification system including: one or more optional heat exchangers for controlling temperature of the gaseous feedstream to temperatures in a range from about 20° C. to about 100° C., adsorption vessels containing particulate beds of a suitable solid adsorbents, and means for analysis of feed and effluent streams, such as an on-line analytical system.

During operation of the integrated olefin purification system, a fluid mixture containing less than about 5000 parts per million by weight of the diene and acetylenc impurities formed by chemical conversions in commercial thermal cracking processes, is, for example hydrocarbons fed from the overhead of a depropanizer distillation tower or intermediate storage, optionally through an acetylene hydrogenation unit and then through a feed exchanger to control temperature during adsorption. Effluent from the feed exchanger flows through the first of two adsorption vessels which contain beds of a suitable solid adsorbents.

During operation the fluid mixture passes though the bed of particulate adsorbent at gas hourly space velocities in a range of from about 0.05 hours⁻¹ to about 20,000 hours⁻¹ and even higher, preferably at gas hourly space velocities in a range of about 0.5 hours⁻¹ to about 10,000 hours⁻¹.

Compositions of the gaseous feed and effluent of each adsorption vessel is monitored by on-line analytical system. While levels of diene and acetylenic impurities in the effluent from the first adsorption vessel in purification service are in a range downward from a predetermined level, the effluent flows through the second vessel and directly to further purification as desired, or to storage. When the level of diene and/or acetylenic impurities in the effluent of a first adsorption vessel in purification service reaches or exceeds the predetermined level, that adsorption vessel is isolated from the process flow, and thereafter the resulting bed of loaded adsorbent is treated to effect release of the contained diene and acetylenic impurities from the adsorbent by hydrogenation. Advantageously the resulting bed of adsorbent in the first vessel is thereafter regenerated in the presence of a reducing gas comprising dihydrogen and containing at least 50 parts per million of carbon monoxide, to effect release of the contained impurities from the adsorbent

Suitable absorbents for used in the first zone have capacity to treat from about 300 to about 40,000 pounds of olefin feed per pound of adsorbent where the olefin feed contains about 0.5 parts per million (ppm) of the diene and acetylenic impurities. Approximately 5×10⁻⁴ pounds of the impurities to about 1×10⁻² pounds are, advantageously, adsorbed per pound of adsorbent before regeneration is required.

During continuous operation of this embodiment, the time required for treating, alternately, of the loaded adsorbent to effect release of the contained impurities from the adsorbent by hydrogenation, is provided by using two (as shown) or more independent adsorption vessels containing beds. Regenerations are, advantageously, performed according to this invention in three steps.

During the first stage of regeneration dry inert gas, such as methane, ethane, or nitrogen which is, preferably, free of carbon oxides, unsaturated hydrocarbons and hydrogen is fed, from, for example a nitrogen gas supply system exchanger to control temperature during regeneration. The dry inert gas flows through the bed of loaded adsorbent thereby purging gaseous hydrocarbons therefrom to disposal.

During the second stage of regeneration a reducing gas stream comprising dihydrogen and containing at least 50 parts per million of carbon monoxide, to effect release of the contained impurities from the adsorbent. Preferably the reducing gas stream comprising predominately dihydrogen containing from about 50 to 500 parts per million of carbon monoxide.

Where heating of the regeneration gas is desired, rates of temperature increase during the second stage of regeneration are, preferably, controlled to rates of less than about 11° C. per minute (about 20° F. per minute) while increasing temperature in the range of from about 4° C. to about 200° C. (about 40° F. to about 400° F.). Pressures of the hydrogen-rich reducing gas during the second stage of regeneration are, advantageously, in a range from about 5 psig to about 500 psig. While the reducing gas is flowing through the adsorbent bed, effluent gas composition is, periodically, monitored with gas analyzer. Second stage of regeneration is complete when C2+ hydrocarbon levels in the effluent gas from the bed have been reduced to C2+ hydrocarbon levels in the feed.

Third stage regeneration involves purging all gaseous hydrogen from the adsorption vessel with an inert gas, e.g. nitrogen with or without a saturated hydrocarbon gas such as methane or ethane, while the vessel is at temperatures in a range upward from about 60° C. (140° F.). During this third stage of regeneration flow of inert gas, at or below ambient temperature and about 5 to about 100 psig, cools the vessel to about ambient temperature thereby completing the regeneration process.

Surface area of adsorbents can be determined by the Brunaur-Emmett-Teller (BET) method or estimated by a simpler Point B method. Adsorption data for nitrogen at the liquid nitrogen temperature, 77 K, are usually used in both methods. The Brunaur-Emmett-Teller equation, which is well known in the art, is used to calculate the amount of nitrogen for mono-layer coverage. The surface area is taken as the area for mono-layer coverage based on the nitrogen molecular area, 16.2 square Angstroms, obtained by assuming liquid density and hexagonal close packing. In the Point B method, the initial point of the straight portion of the Type II isotherm is taken as the completion point for the mono-layer. The corresponding amount adsorbed multiplied by molecular area yields the surface area.

Dispersion and surface area of active metal sites was determined by carbon monoxide chemisorption using a Pulse Chemisorb 2700 (Micromeritics). In this procedure, approximately 4 gram samples were purged with helium carrier gas, calcined in air at 500° C. for 1 hr, purged with helium, reduced in hydrogen at 500° C., purged with helium, and cooled to room temperature. The sample was treated with 49.5 percent carbon monoxide in helium and the dosed with 0.045 mL pulses of 49.5 percent carbon monoxide (CO), balance nitrogen, and the carbon monoxide uptake was measured by a thermal conductivity cell. Palladium dispersion values were calculated assuming one carbon monoxide molecule per palladium atom. Palladium loadings are weight percent palladium metal.

In characterizing the pore volume, both total pore volume and its distribution over the pore diameter are needed. The total pore volume is usually determined by helium and mercury densities or displacements. Helium, because of its small atomic size and negligible adsorption, gives the total voids, whereas mercury does not penetrate into the pores at ambient pressure and gives inter-particle voids. The total pore volume equals the difference between the two voids.

Palladium on a high-surface-area γ-Al₂O₃ is a preferred adsorbent for purification of olefins in accordance with this invention. In order to introduce palladium and/or other suitable metal ions on a high-surface-area γ-Al₂O₃, any known technique for monolayer dispersion can be employed. The phenomenon of spontaneous dispersion of metal oxides and salts in monolayer or submonolayer forms onto surfaces of inorganic supports with high surface areas has been studied extensively in the literature (e.g., Xie and Tang, 1990).

### EXAMPLES OF THE INVENTION

The following examples will serve to illustrate certain specific embodiments of the herein disclosed invention. These examples should not, however, be construed as limiting the scope of the novel invention as there are many variations which may be made thereon without departing from the spirit of the disclosed invention, as those of skill in the art will recognize.

A 50 mL TEFLON-lined stainless steel pressure vessel was loaded with a commercially available adsorbent (about 43 mL of 0.5 percent palladium on γ-Al₂O₃), and a centrally disposed thermocouple system to monitor bed temperatures. After this adsorption vessel was connected into a gas adsorption unit which provided required control of feed gases, temperatures, pressures, and analytical means, the adsorbent bed was run in the down-flow mode. Nitrogen was purged through the vessel before reducing the oxidized PdO/γ-Al₂O₃ adsorbent by heating to 49° C. in a flow of hydrogen. Electrical heating tape wrapped around the vessel was used to supply heat needed during reduction.

Initially, the cell was de-pressurized, purged with nitrogen, and pressurized to 200 psig with source of ethylene, or 15 psig with source of propylene, “spiked” with propadiene and/or one or more acetylenic impurity. Olefin flow rates were measured with a bubble meter. Periodically, a portion of the effluent was injected into a gas chromatograph (GC) to determine concentrations of the components. After an impurity had broken through the adsorbent (defined as 1 ppm measured on the GC trace), one or two additional samples were taken and impurity levels determined. These data were then plotted on a graph of impurity concentration versus time. The concentration of the impurity was then extrapolated back to the zero impurity to determine the time-to-breakthrough. The amount of adsorbed impurity was calculated from the feed flow rate, the concentration of the impurity in the feed stream, and the time to breakthrough.

### Example 1

This example of the invention demonstrated the use of an adsorption bed at 49° C., as described above, in purification of an impure ethylene stream contaminated with 203 ppm of propadiene. The level of propadiene in the effluent was below detectable limits until breakthrough. The capacity of the 0.5 percent palladium on γ-Al2O₃ adsorbent was 0.3 mL of propadiene/mL of adsorbent.

### Examples 2 and 3

These examples of the invention demonstrated the use of an adsorption bed, as described above, in purification of an impure propylene stream contaminated with 209 ppm of propadiene. The level of propadiene in the effluent was below detectable limits until breakthrough. The average capacity of the adsorbent for Examples 2 and 3 was 0.8 mL of propadiene/mL of adsorbent. These multiple runs demonstrated that the adsorbent was regenerated in accordance with the invention.

### Examples 4-7

These examples of the invention demonstrated the use of an adsorption bed, as described above, in purification of an impure ethylene stream contaminated with 50 ppm of propadiene, 50 ppm of methyl-acetylene, and 100 ppm of acetylene. The level of propadiene in the effluent was below detectable limits until breakthrough. The average capacity of the adsorbent for Examples 4-7 was 0.13 mL of propadiene/mL of adsorbent, 0.13 mL of methyl-acetylene/mL of adsorbent, and 0.36 mL of acetylene/mL of adsorbent,.

### Examples 8 and 9

These examples of the invention demonstrated the use of an adsorption bed, as described above, in purification of an impure ethylene stream contaminated with 200 ppm of methyl-acetylene. The level of methyl-acetylene in the effluent was below detectable limits until breakthrough. The average capacity of the adsorbent for Examples 8 and 9 was 0.29 mL of methyl-acetylene/mL of adsorbent.

### Example 10

This example of the invention demonstrated the use of an adsorption bed, as described above, in purification of an impure propylene stream contaminated with 221 ppm of methyl-acetylene. The level of methyl-acetylene in the effluent was below detectable limits until breakthrough. The capacity of the 0.5 percent palladium on γ-Al₂O₃ adsorbent was 0.42 mL of methyl-acetylene/mL of adsorbent.

Examples have been presented and hypotheses advanced herein in order to better communicate certain facets of the invention. The scope of the invention is determined solely by the scope of the appended claims.

For the purposes of the present invention, “predominantly” is defined as more than about ninety per cent. “Substantially” is defined as occurring with sufficient frequency or being present in such proportions as to measurably affect macroscopic properties of an associated compound or system. Where the frequency or proportion for such impact is not clear substantially is to be regarded as about twenty per cent or more. The term “Essentially” is defined as absolutely except that small variations which have no more than a negligible effect on macroscopic qualities and final outcome are permitted, typically up to about one percent.

That which is claimed is:

1. A process for purification of olefins which comprises:
providing a fluid mixture predominantly comprising at least one olefin of from 2 to about 8 carbon atoms, impurities comprising propadiene and optionally hydrocarbon compounds of from 3 to about 6 carbon atoms having more than one double bond and/or acetylenic impurities having the same or similar carbon content in an amount of up to about 1 percent by volume base upon the total amount of olefin present and optionally saturated hydrocarbon gases;
passing the fluid mixture through a particulate bed of adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditions suitable for adsorption within the bed, to effect, in the presence of an essentially dihydrogen-free atmosphere within the bed, selective adsorption and/or complexing of the contained impurities with the adsorbent, and thereby obtain purified effluent which contains less than about 1 part per million by volume of the propadiene impurity; and
thereafter regenerating the resulting bed of adsorbent in the presence of a reducing gas comprising dihydrogen to effect release of the contained impurities from the adsorbent.

2. The process according to claim 1 wherein the adsorbent further comprises at least one element selected from the group consisting of lithium, sodium, potassium, zinc, molybdenum, tin, tungsten, and iridium, dispersed on the support material.

3. The process according to claim 1 wherein the support is a material selected from the group consisting of alumina, silica, active carbon, clay and zeolites, and has surface area in a range of from about 10 to about 2,000 square meters per gram as measured by the BET gas adsorption method.

4. The process according to claim 3 wherein the metal dispersed on the support material is at least one element selected from the group consisting of iron, cobalt, nickel, copper, palladium, silver and platinum, and the absorbent has a dispersed metal content in a range of from about 0.01 to about 10 percent based on the total weight of the adsorbent.

5. The process according to claim 4 wherein the fluid mixture passes though the bed of particulate adsorbent at gas hourly space velocities in a range of from about 0.05 hours⁻¹ to about 20,000 hours⁻¹ measured at standard conditions of 0° C. and 760 mm Hg.

6. The process according to claim 1 wherein the adsorbent comprises at least about 90 weight percent of a gamma alumina having surface area in a range of from about 80 to about 500 square meters per gram as measured by the BET gas adsorption method.

7. The process according to claim 6 wherein the metal dispersed on the support material is palladium, and the absorbent has a palladium content in a range of from about 0.01 to about 10 percent based on the total weight of the adsorbent.

8. The process according to claim 1 wherein the olefin in the fluid mixture being purified is predominantly ethylene or propylene, the fluid mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range of from about negative 5° C. to about 65° C.

9. The process according to claim 8 wherein the adsorbent comprises at least about 90 weight percent of a gamma alumina having surface area in a range of from about 150 to about 350 square meters per gram as measured by the BET gas adsorption method, and wherein the metal dispersed on the support material is palladium, and the absorbent has a palladium content in a range of from about 0.01 to about 10 percent based on the total weight of the adsorbent.

10. The process according to claim 1 wherein the adsorbent has a metal dispersion value of at least 10 percent as measured by carbon monoxide chemisorption method.

11. A process for purification of olefins produced by thermal cracking of hydrocarbons which comprises:
passing a fluid mixture comprising at least about 99 percent by volume of an olefin having from 2 to about 4 carbon atoms, and impurities comprising propadiene and optionally hydrocarbon compounds of from 3 to about 6 carbon atoms having more than one double bond and/or acetylenic impurities having the same or similar carbon content in an amount in a range upward from about 1 to about 5000 parts per million by volume, through a particulate bed of adsorbent comprising predominantly a support material selected from the group alumina, silica, active carbon, clay and zeolites having surface area in a range of from about 10 to about 2,000 square meters per gram as measured by the BET gas adsorption method, on which is dispersed at least one metallic element selected from the group consisting of chromium, iron, cobalt, nickel, copper, ruthenium, palladium, silver and platinum, to provide an effluent stream from the bed;
effecting, in the presence of and essentially dihydrogen-free atmosphere within the bed, selective and reversible adsorption and/or complexing of the contained diene and acetylenic impurities with the adsorbent, until levels of the diene impurities in the effluent stream increase to a predetermined level in a range downward from about 1 parts per million by volume; and
thereafter regenerating the resulting bed of adsorbent in the presence of a reducing gas comprising dihydrogen to effect release of the contained diene impurities from the adsorbent.

12. The process according to claim 11 wherein the adsorbent further comprises at least one element selected from the group consisting of lithium, sodium, potassium, zinc, molybdenum, tin, tungsten, and iridium, dispersed on the support material.

13. The process according to claim 11 wherein the support is a material selected from the group consisting of alumina, silica, carbon clay and zeolites, and has surface area in a range of from about 10 to about 2,000 square meters per gram as measured by the BET gas adsorption method.

14. The process according to claim 13 wherein the metal dispersed on the support material is at least one element selected from the group consisting of iron, cobalt, nickel, copper, palladium, silver and platinum,, and the absorbent has a dispersed metal content in a range of from about 0.01 to about 10 percent based on the total weight of the adsorbent.

15. The process according to claim 14 wherein the fluid mixture passes though the bed of particulate adsorbent at space velocities in a range of from about 0.05 hours⁻¹ to about 20,000 hours₋₁ measured at standard conditions of 0° C. and 760 mm Hg

16. The process according to claim 11 wherein the adsorbent comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur.

17. The process according to claim 16 wherein the metal dispersed on the support material is palladium, and the absorbent has a palladium content in a range of from about 0.01 to about 10 percent based on the total weight of the adsorbent.

18. The process according to claim 11 wherein the olefin in the fluid mixture being purified is predominantly ethylene or propylene, the fluid mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range of from about negative 35° C. to about 65° C.

19. A process for purification of an olefinic stream to obtain a diene-free feedstock suitable for formation of a polymeric resin, which purification process comprises:
providing an impure gaseous stream comprising at least about 99 percent by volume of an olefin selected from the group consisting of ethylene and propylene, impurities comprising propadiene and optionally hydrocarbon compounds of from 3 to about 5 carbon atoms having more than one double bond and/or acetylenic impurities having the same or similar carbon content in an amount in a range upward from about 1 to about 3500 parts per million by volume based upon the total amount of olefin present and optionally saturated hydrocarbon gases;
passing the impure stream through a bed of adsorbent which is free of a substantial amount of carbon monoxide, the adsorbent comprising at least about 90 weight percent of gamma alumina having surface area in a range of from about 150 to about 350 square meters per gram as measured by the BET gas adsorption method, on which is dispersed at least one element selected from the group consisting of iron, cobalt, nickel, copper, palladium, silver and platinum, in the zero valent state, to effect, under conditions suitable for adsorption within the bed, selective adsorption and/or complexing of the contained impurities with the adsorbent, thereby obtaining an effluent steam of feedstock which contains less than about 0.5 parts per million by volume of carbon monoxide and less than about 1 parts per million by volume of propadiene;
effecting, in the presence of an essentially dihydrogen-free atmosphere within the bed, selective adsorption and/or complexing of the contained impurities with the adsorbent, until levels of the impurities in the effluent stream increase to a limiting level in a range downward from about 1 parts per million by volume; and
thereafter regenerating the resulting bed of adsorbent in the presence of a reducing gas comprising dihydrogen which reducing gas is free of a substantial amount of carbon monoxide, to effect release of the contained impurities from the adsorbent.

20. The process according to claim 19 wherein the adsorbent comprises at least about 90 weight percent of a gamma alumina having surface area in a range of from about 150 to about 350 square meters per gram as measured by the BET gas adsorption method, the metal dispersed on the support material is palladium, and the absorbent has a palladium content in a range of from about 0.01 to about 10 percent based on the total weight of the adsorbent, and wherein the gaseous mixture, while passing through the bed, is at temperatures in a range of from about negative 5° C. to about 65° C.
